(19) United States
(12) Patent Application Publication
(10) Pub. No.: US 2008/0027139 A1
(43) Pub. Date: Jan. 31, 2008

(54) Use of Alpha-Ketoglutarate and Related Compounds

(76) Inventors: Stefan G. Pierzynowski, Lund (SE); Radoslaw Piotr Radzki, Lublin (PL); Marek Bienko, Lublin (PL)

Correspondence Address: LADAS & PARRY LLP, 224 SOUTH MICHIGAN AVENUE, SUITE 1600, CHICAGO, IL, 60604, US

(21) Appl. No.: 11/629,398

(22) PCT Filed: Jun. 16, 2005
(86) PCT No.: PCT/SE05/00929

§ 371 Date: May 22, 2007

(30) Foreign Application Priority Data
Jun. 17, 2004 (PL) 368572

(51) Int. Cl. A61K 31/194; A61K 31/195; A61P 3/00

(52) U.S. Cl. 514/574; 514/561

(57) ABSTRACT

The use of at least one member selected from alpha-ketoglutaric acid, glutamine, glutamic acid and pharmaceutically acceptable salts of these acids, amides of alpha-ketoglutaric acid with an amino acid or di- or tripeptide, dipeptides of glutamine and another amino acid, tripeptides of glutamine and other amino acids, dipeptides of glutamine acid and other amino acids, tripeptides of glutamic acid and other amino acids and pharmaceutically acceptable salts of dipeptides and tripeptides, pharmaceutically accepted physical mixtures of alpha-ketoglutaric acid or a pharmaceutically acceptable salt thereof and at least one amino acid, for the manufacture of a pharmaceutical preparation or a food or a feed supplement for the treatment or prophylaxis of a condition of increased plasma levels of at least one member selected from cholesterol, low density lipids (LDL) and glycerides or for the promotion of high density lipid (HDL) production.

The present invention relates to a new use of known pharmacologically active chemical compounds. More particularly, the present invention relates to the new use of certain acids, lipids and salts and mixtures thereof for the manufacture of a pharmaceutical preparation or a food or feed supplement for the treatment or prophylaxis of a condition of increased plasma levels of at least one member selected from the group consisting of cholesterol, low density lipids (LDL) and glycerides or for the promotion of high density lipid (HDL) production in vertebrates such as birds, and mammals, including man.

Cholesterol is an amphipatic lipid and as such it is an essential structural component of the biological membranes and of the outer layer of plasma lipoproteins. Lipoproteins transport free cholesterol in the bloodstream where it is exchanged, on the principle of balance, with the cholesterol contained in other lipoproteins and plasma. Esterized cholesterol is a buffer cholesterol found in most tissues of the body. It is transported as “a load” in the core of plasma lipoproteins. LDL, low density lipoprotein, acts as an intermediary in transferring cholesterol and cholesterol esters to many tissues. Free cholesterol is removed from tissues by HDL, high density lipoprotein, and transported to the liver where it is metabolized into bile acids, and it is finally removed from the body in the process of the reverse cholesterol transport. Cholesterol is also the main component of gallstones. However, its most important role in pathological processes is its active participation in atherosclerosis of blood vessels, which leads to diseases of cerebral, coronary and systemic arteries. The intensification of arteriosclerosis correlates positively with a high LDL to HDL concentration ratio, as HDL is a specific cholesterol “sweeper” during its transport from tissues to the liver.

Cholesterol is a precursor of all other steroids in the body such as corticosteroids, sex hormones, bile acids, and vitamin D. It is a typical product of animal metabolism; it follows that it is found in foods of animal origin such as the egg yolk, meat, liver and brain.

The adult body contains approximately 140 g of the entire (free and esterized) cholesterol of which approximately 40 g is found in the nervous tissue; the remaining 5% is found in plasma. The content of cholesterol in other organs and tissues fluctuates with the most significant changes in the adipose tissue and in the liver.

The deposition of cholesterol esters and other lipids in the connective tissue of artery walls is characteristic to arteriosclerosis. With time cholesterol deposits in arteries undergo hardening, which makes them narrower and impedes or even totally blocks blood flow. When arteries are constricted, insufficient blood flow translates into a deficit of oxygen. The heart is anoxiated (ischaemia occurs), which, in turn, causes chest pain. When one of the coronary arteries is totally blocked, myocardial infarction or heart necrosis develops.

A high level of cholesterol in itself does not trigger any symptoms; therefore, many people are not aware of the harmful effects of its high concentration in the body. An effective decrease in high cholesterol concentration reduces the risk of a coronary disease, heart failure and cardiac death. Besides, a reduction of cholesterol in people who suffer from coronary diseases have experienced myocardial infarction diminishes the risk of another infarction and extends their life span. A decrease in the level of cholesterol concerns all people in all age groups.

Low density lipoproteins or LDL in plasma, which are easily modified during oxidative processes, constitute an important factor in the development of the arteriosclerosis. The beginning of arteriosclerosis is invariably connected with the oxygenation of LDL. Oxygenated LDL (oxyLDL) is commonly considered to be a proartetiosclerotic agent. The oxygenation of LDL consists in peroxidation of the residues of unsaturated fatty acids contained in phospholipids and cholesterol esters. The process is induced by oxygenic free radicals. Macrophages and smooth myocytes take over modified LDL and turn themselves into foam (xanthoma) cells loaded with cholesterol and lipids being the main component of the atheromatous plaque. The formation of foam cells is intensified with an increase in the concentration of plasma in oxyLDL. The thrombocytes aggregation, often leading to intravascular thrombosis, also plays an important role in the pathogenesis of arteriosclerosis.

The atheromatous plaque undergoes specific mineralization (calcification) processes similar to the formation of the osseous tissue. Calcification increases the risk of myocardial infarction irrespective of the patient's age. The complications after angioplasty, e.g. the dissection of the wall of the coronary artery, become more frequent. The calcification also facilitates the formation of an unstable coronary disease, since ruptures at the edges of atheromatous plaque are more common when the stiff plaque is close to the elastic wall of the artery. Calcification also exerts an influence on the tension of the vascular wall and decrease its elasticity making it difficult for arteries to increase their vertical section. The loss of the elasticity can significantly impair hemodynamics and is conducive to the development of heart diseases.

Circulatory system diseases can take one of the following forms of irregularities: an increase in the level of VLDL (mainly triglycerides) at the standard level of LDL, an increase in the level of LDL at the standard level of VLDL (triglycerols) or an increase in the levels of both lipoprotein fractions (cholesterol+triglycerides).

Various external factors may also have an effect on cholesterol concentration. First and foremost, its concentration changes with age. Menopause, which is the effect of the termination of the activity of ovaries or endocrine glands, is of special pertinence. Similar symptoms appear during the course of andropause. Pregnancy increase the concentration of cholesterol is the blood. Fluctuation relating to the menstrual cycle are commonly reported. During ovulation the plasma cholesterol concentration decreases. After ovariectomy or as a result of the natural termination of ovary activity, the concentration of cholesterol and triglycerides increases. Estrogens increase HDL concentration in the blood. Hence, lack of the same translates into a greater risk of arteriosclerosis, since the absence of HDL triggers increased concentration of LDL, and, consequently, an increase risk of circulatory diseases. Oral administration of contraceptives can also increase, notably among younger women, the level of cholesterol.

A bad diet is yet another reason behind elevated levels of cholesterol. The consumption of fatty and highly processed foodstuffs and a decreased consumption of vegetables and fruits makes cholesterol rise in the blood. Proneness to obesity and abnormal, especially too high body weight, can also have an effect on the level of cholesterol and triglycerides. Such persons are reported to have increased amounts of these elements in their bodies. Besides changes in the circulatory system, overweight people are exposed to problems relating to the osseous-skeletal system. Dysarthrosis is likely to occur, and young people whose skeletal system continues to grow, may suffer bone damages, since their bones are not well adapted to carry excessive weight of their bodies.

In accordance with the present invention it was surprisingly found that alpha-ketoglutaric acid, glutamine and glutamic acid and salts and dipeptides and tripeptides of said amino acids and salts, amides and mixtures of alpha-ketoglytaric acid with amino acids may be used for the treatment or prophylaxis of a condition of increased plasma levels of cholesterol, LDL and/or glycerides or for the promotion of HDL production in vertebrates such as birds and mammals, including man.

Thus according to one aspect of the present invention, there is provided the new use of at least one member selected from the group consisting of alpha-ketoglutaric acid, glutamine, glutamic acid and pharmaceutically acceptable salts of these acids, amides of alpha-ketoglutaric acid and an amino acid or a di- or tripeptide dipeptides of glutamine and another amino acid, tripeptides of glutamine and other amino acids, dipeptides of glutamine acid and other amino acids, tripeptides of glutamic acid and other amino acids and pharmaceutically acceptable salts of said dipeptides and tripeptides, pharmaceutically accepted physical mixtures of alpha-ketoglutaric acid or a pharmaceutically acceptable salt thereof and at least one amino acid for the manufacture of a pharmaceutical preparation or a food or feed supplement for the treatment or prophylaxis of a condition of increased plasma levels of at least one member selected from the group consisting of cholesterol, low density lipids (LDL) and glycerides or for the promotion of high density lipid (HDL) production in vertebrates such as birds and mammals, including man.

According to a preferred embodiment of the invention alpha-ketoglutaric acid or an alkali or alkaline earth metal salt thereof or a combination thereof is used. Preferably sodium alpha-ketoglutarate is used.

According to another aspect of the present invention there is provided a method for the treatment or prophylaxis of a condition of increased plasma levels of at least one member selected from the group consisting of cholesterol, low density lipids (LDL) and glycerides in birds and mammals, including man, which method comprises administering to a subject in need for such treatment or prophylaxis of an effective plasma level lowering amount of at least one member selected from the group consisting of alpha-ketoglutaric acid, glutamine, glutamic acid and pharmaceutically acceptable salts of these acids, amides of alpha-ketoglutaric acid and an amino acid or a di- or tripeptide, dipeptides of glutamine and another amino acid, tripeptides of glutamine and other amino acids, dipeptides of glutamic acid and other amino acids, tripeptides of glutamic acid and other amino acids and pharmaceutically acceptable salts of said dipeptides and tripeptides, pharmaceutically accepted physical mixtures of alpha-ketoglutaric acid or a pharmaceutically acceptable salt thereof and at least one amino acid.

According to a further aspect of the present invention there is provided a method for the promotion of high density lipid (HDL) production in a bird or a mammal, including man, which method comprises administering to said bird or mammal an effective plasma HDL level increasing amount of at least one member selected from the group consisting of alpha-ketoglutaric acid, glutamine, glutamic acid and pharmaceutically acceptable salts of these acids, amides of alpha-ketoglutaric acid and an amino acid or a di- or tripeptide, dipeptides of glutamine and another amino acid, tripeptides of glutamine and other amino acids, dipeptides of glutamine acid and other amino acids, tripeptides of glutamic acid and other amino acids and pharmaceutically acceptable salts of said dipeptides and tripeptides, pharmaceutically accepted physical mixtures of alpha-ketoglutaric acid a or a pharmaceutically acceptable salt thereof and at least one amino acid.

According to preferred embodiments of these aspects alpha-ketoglutaric acid or an alkali or alkali or alkaline earth metal salt thereof or a combination thereof is administered. Most preferably sodium alpha-ketoglutarate is administered.

The food or feed supplements and the pharmaceutical preparations of the active principle or principles used in accordance with the present invention may be administered to a vertebrate, including mammals and birds, such as rodent, such as a mouse, rat, guinea pig, or a rabbit; a bird, such as a turkey, hen or chicken and other broilers and free going animals; a cow, a horse, a pig or piglet and other farm animals, a dog, a cat and other pets, and in particular humans.

Administration may be performed in different ways depending on what species of vertebrate to treat, on the condition of the vertebrate in the need of said methods, and the specific indication to treat.

In one embodiment, the administration is done as a food or feed supplement, such as a dietary supplement and/or a component in form of solid food and/or beverage. Further embodiments may be in suspensions or solutions, such as a beverage further described below. Also, the formats may be in capsules or tablets, such as chewable or soluble, e.g. effervescent tablets, as well as powder and other dry formats known to the skilled man in the art, such as pellets, such as micropellets, and grains.

The administration may be as a parenteral, rectal or oral food or feed supplement, as revealed above. Parenteral vehicles include sodium chloride solution, Ringer's dextrose, dextrose and sodium chloride, lactated Ringer's or fixed oils.

The food and feed supplement may also be emulsified. The active therapeutic ingredient or ingredients may then be mixed with excipients, which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH, buffering agents, which enhance the effectiveness of the active ingredient.

Different formats of the oral food or feed supplement may be supplied, such as solid food, liquids or lyophilized or otherwise dried formulations. It may include diluents of various buffers (e.g., Tris-HCl, acetate, phosphate), pH and ionic strength, additives such as albumin or gelatine to prevent absorption to surfaces, detergents (e.g., Tween 20, Tween 80, Pluronic F68, bile acid salts). solubilizing agents (e.g., glycerol, polyethyleneglycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the composition, complexation with metal ions, or incorporation of the material into or onto particulate preparations of polymeric compounds such as polylactic acid, polglycolic acid, hydrogels, etc, or onto liposomes, microemulsions, micelles, unilamellar or multilamellar-vesicles, erythrocyte ghosts, or spheroplasts. In one embodiment, the food or feed supplement is administered in the form of a beverage, or a dry composition thereof, in any of the methods according to the invention.

The beverage comprises an effective amount of the active ingredient or ingredients thereof, together with a nutritionally acceptable water-soluble carrier, such as minerals, vitamins, carbohydrates, fat and proteins. All of these components are supplied in a dried form if the beverage is provided in a dry form. A beverage provided ready for consumption further comprises water. The final beverage solution may also have a controlled tonicity and acidity, e.g. as a buffered solution according to the general suggestions in the paragraph above.

The pH is preferably in the range of about 2-5, and in particularly about 2-4, to prevent bacterial and fungal growth. A sterilised beverage may also be used, with a pH of about 6-8.

The beverage may be supplied alone or in combination with one or more therapeutically effective composition.

According to a further embodiment the pharmaceutical preparations as drugs for oral and rectal use may be in the form of tablets, lozenges, capsules, powders, aqueous or oily suspensions, syrups, elixirs, aqueous solutions and the like comprising the active ingredient or ingredients in admixture with a pharmaceutically acceptable carrier and/or additives, such as diluents, preservatives, solubilizers, emulsifiers, adjuvants and/or carriers useful in the methods and use disclosed in the present invention.

Further, as used herein “pharmaceutically acceptable carriers” are well known to those skilled in the art and may include, but are not limited to, 0.01-0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/-aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

Amino acids forming part of amides with alpha-ketoglutaric acid or of dipeptides with glutamine or glutamic acid or tripeptides with glutamine and/or glutamic acid may be any of the amino acids occurring as components in peptides in nature. The same applies to the pharmaceutically accepted physical mixtures of alpha-ketoglutaric acid or salts thereof with at least one amino acid. Preferably the amino acid or acids is/are selected from the group consisting of arginine, ornithine, leucine, isoleucine and lysine.

Said amino acids are preferably used in their L-configuration.

Examples of amides of alpha-ketoglutaric acid with an amino acid or a di- or tripeptide include, but are not limited to, amides of alpha-ketoglutaric acid with an amino acid selected from the group consisting of glutamine, glutamic acid, arginine, ornithine, lysine, proline, isoleucine and leucine and amides of alpha-ketoglutaric acid with a dipeptide of glutamine and any of glutamic acid, arginine, ornithine, lysine, proline, isoleucine and leucine and with a dipeptide of glutamic acid and any of arginine, ornithine, lysine, proline, isoleucine and leucine.

Examples of di- and peptides of glutamine and glutamic acid with other amino acids include those mentioned above in connection with amides of alpha-ketoglutaric acid with di- or tripeptides.

Examples of physical mixtures of α-ketoglutaric acid or salts thereof with at least one amino acid includes, but are not limited to, physical mixtures of at least one member selected from the group consisting of alpha-ketoglutaric acid and the sodium, potassium, calcium and magnesium salts thereof with any of glutamine, glutamic acid, arginine, ornithine, leucine, isoleucine, lysine and proline and any combinations of said amino acids.

The molar ratio of alpha-ketoglutaric acid or salts thereof to amino acid or amino acids of said physical mixtures will in general be within the limits of from 1:0.01 to 1:2, preferably from 1:0.1 to 1:1.5 and most preferably from 1:0.2 to 1:1.0.

The dosage to be administered will vary depending on the active principle or principles to be used, the condition to be treated, the age, sex, weight etc. of the patient to be treated but will generally be within the range from 1 to 1000 mg/kg bodyweight/day, preferably from 10 to 100 mg/kg bodyweight/day.

The invention will now be further illustrated by means of a number of examples which should not be construed to limit the scope of the invention.

### EXAMPLE 1

A study was conducted on Wistar female rats kept in standard weather conditions. During the first 14 days the rats were subjected to a period of acclimation to the conditions of the animal room (temperature 22° C.±2° C., 12 hrs/12 hrs day/night cycle, humidity 55%±5%) and had permanent access to water and were fed with the standard rat pelleted diet (LSM) (Wytwórnia Pasz [fodder producing facility] “Agropol” Motycz near Lublin). Afterwards the female rates were randomly divided into 2 groups: ovariectomy were performed in all animals in group one (OVX) and placebo operation were done in the second group (SHO). Surgical procedures were performed under general anesthesia (rometare, ketamine and atropine in doses 2, 10 and 0.05 mg/kg of bodily weight respectively.

Studies were conducted on the animals from the two groups: females received experimental solutions from the seventh day following the operation (simulation of the first period of the lack of activity of the ovarian hormones) (Experiment I 1), females aged 7 months with a 5 month period in which there was no activity of hormonal ovaries (simulation of the period with a prolonged absence of the activity of ovarian hormones) (Experiment I 2) (Tab. 2). The animals in each age group were divided into groups which received per os the basic solution (Tab. 1) and the basic solution which was thinned tenfold and one hundredfold (hereinafter referred to as the AKG 1, 0.1, and 0.01 doses).

In each of Experiments I1 and I2 40 animals was used for each experimental doses making a total of 240 animals.

TABLE 1
Composition of the basic placebo and alpha-ketoglutaric
acid (AKG) experimental solutions.

| Composition | Placebo | Experimental Soluti [symbol] |
|---|---|---|
| AKG | — | 146 g |
| Glucose | 300 g | 300 g |
| Sucrose | 150 g | 150 g |
| NaOH | 36 g | 36 g |
| KOH | 7.5 g | 7.5 g |
| Ca(OH)2 | 4.6 g | 4.6 g |
| Mg(OH)2 | 1.8 g | 1.8 g |
| Water | up to 10 l (pH 4.6) | up to 10 l (pH 4.6) |

TABLE 2
Diagram of Experiment 1

| | |
|---|---|
| Placebo | AKG |

Experiment No. I-1
Experiment commenced 7 days after surgical operations

| | | |
|---|---|---|
| SHO | n = 10 | n = 10 |
| OVX | n = 10 | n = 10 |

Experiment No. I-2
Experiment commenced 5 months after surgical operations

| | | |
|---|---|---|
| SHO | n = 10 | n = 10 |
| OVX | n = 10 | n = 10 |

Blood samples were collected immediately after slaughter, and the plasma removed by centrifugation was stored in a safe place for further biochemical analyses.

Biochemical Analyses

The entire cholesterol concentration was determined by means of commonly available analytical equipment using the spectrophotometric method.

The results are reported in Tables 3 to 8 below.

TABLE 3
Protective effect of dose 0.01 of AKG on cholesterol
concentration (mmol/l) in blood serum (7 days after
surgery + 60 days of AKG treatment)

| | SHO + | SHO + AKG | OVX + | OVX + AKG |
|---|---|---|---|---|
| | PLACEBO | (dose 0.01) | PLACEBO | (dose 0.01) |
| FEMALE | 1.45 ± 0.08 | 1.50 ± 0.19 | 1.70 ± 0.08^{a} | 1.46 ± 0.08^{b} |

( X̅ ± S.E.M
(^{a,b,}p < 0.05)
(SHO = sham-operated; OVX = ovariectomized Wistar rats)

TABLE 4
Protective effect of dose 0.1 of AKG on cholesterol
concentration (mmol/l) in blood serum (7 days after
surgery + 60 days of AKG treatment)

| | SHO + | SHO + AKG | OVX + | OVX + AKG |
|---|---|---|---|---|
| | PLACEBO | (dose 0.01) | PLACEBO | (dose 0.01) |
| FEMALE | 1.37 ± 0.08 | 1.45 ± 0.19 | 1.56 ± 0.08 | 1.47 ± 0.08 |

(SHO = sham-operated; OVX = ovariectomized Wistar rats)

TABLE 5
Therapeutic effect of doses 0.01 of AKG on cholesterol concentration
(mmol/l) in blood serum (150 days after surgery + 60 days
of AKG treatment)

| | SHO + | SHO + AKG | OVX + | OVX + AKG |
|---|---|---|---|---|
| | PLACEBO | (dose 0.01) | PLACEBO | (dose 0.01) |
| FEMALE | 1.87 ± 0.13 | 1.59 ± 0.06^{a} | 2.04 ± 0.06^{bA} | 1.68 ± 0.09^{B} |

( X̅ ± S.E.M)
(^{a,b,}p < 0.01)
(^{A,B}p < 0.05) OVX = ovariectomized
(SHO = sham-operated; Wistar rats)

TABLE 6
Therapeutic effect of dose 0.1 of AKG on cholesterol concentration
(mmol/l) in blood serum (150 days after surgery + 60 days of AKG
treatment)

| | SHO + | SHO + AKG | OVX + | OVX + AKG |
|---|---|---|---|---|
| | PLACEBO | (dose 0.1) | PLACEBO | (dose 0.1) |
| FEMALE | 1.61 ± 0.07 | 1.60 ± 0.08^{a} | 1.88 ± 0.057^{bA} | 1.59 ± 0.06^{B} |

( X̅ ± S.E.M)
(^{a,b}p < 0.05)
(^{A,B}p < 0.01)

TABLE 7
Protective effect of dose 1.0 of AKG on cholesterol
concentration (mmol/l) in blood serum (7 days after
surgery + 60 days of AKG treatment)

| | SHO + | SHO + AKG | OVX + | OVX + AKG |
|---|---|---|---|---|
| | PLACEBO | (dose 1.0) | PLACEBO | (dose 1.0) |
| FEMALE | 1.56 ± 0.25 | 1.28 ± 0.11 | 1.70 ± 0.12 | 1.44 ± 0.11 |

TABLE 8
Therapeutic effect of dose 1.0 of AKG on cholesterol
concentration (mmol/l) in blood serum (90 days after
surgery + 60 days of AKG treatment)

| | SHO + | SHO + AKG | OVX + | OVX + AKG |
|---|---|---|---|---|
| | PLACEBO | (dose 1.0) | PLACEBO | (dose 1.0) |
| FEMALE | 1.15 ± | 0.76 ± 0.06^{b} | 1.92 ± 0.04^{A#} | 0.49 ± 0.09^{B}* |
| | 0.08^{a #}* | | | |

(^{a,b}p < 0.05; ^{A,B}p < 0.001 statistically significant differences between placebo vs. AKG treated groups)
(#p < 0.001; *p < 0.01 statistically significant differences between SHO PLACEBO vs. OVX treated groups respectively).

### EXAMPLE 2

### Experiment II

A study was conducted on Wistar rats (84 animals being used in total) with an average body weight of 250 g. subjected to a period of acclimation to the conditions of the animal room (temperature 22° C.±2° C., 12 hrs/12 hrs day/night cycle, humidity 55%±5%) and had permanent access to water and food. Afterwards all animals received fodder enriched with 1% cholesterol (Sigma-Aldrich, Germany) and 10% lard in order to obtain hypercholesterolaemia.
12 rats (6 females and 6 males) were killed on day 0 as an introduction control.
12 rats (6 females and 6 males) were killed at day 30.
12 rats (6 females and 6 males) were killed at day 60.

Remaining 48 rats were divided into 3 groups and the experiment continued for another 60 days on cholesteremic diet:
1. Placebo group—24 rats (12 females and 12 males)
2. 1 g AKG group—12 rats (6 females and 6 males)
3. 0.1 g AKG group—12 rats (6 females and 6 males)

The placebo and experimental solutions were as set forth in Example 1.

TABLE 9
Diagram of Experiment II

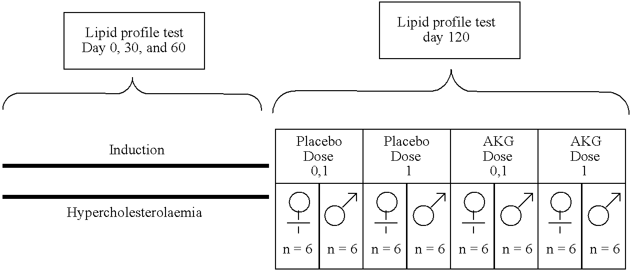

Biochemical Analyses

During the first 60 days of using cholesterol and lard enriched diet, the lipid profile was checked three times to determine the concentration of the entire cholesterol, the fraction of LDL and HDL and triglycerides. Measuring tests were performed a day before the said diet was commenced (day 0) and subsequently after 30 and 60 days of the application of the said diet. On the sixty first day of the experiment animals were divided into two groups: one receiving the placebo solution and the other receiving AKG solution (AKG in doses 0.1 and 1 (Table 9). All the time animals continued to receive experimental fodder. During the period when placebo and AKG solutions were administered, the lipid profile was checked on the 120^{th} day of the experiment.

Blood plasma was used in all cases and the tests were conducted immediately after slaughter.

The results are reported in Tables 10 to 14 below

TABLE 10
Total cholesterol level (mg/dl) in the blood serum of rats

| | | | | | 120 day + | 120 day + |
|---|---|---|---|---|---|---|
| | | | | 120 day + | AKG | AKG |
| | 0 day | 30 day | 60 day | H₂O | (dose 0.1) | (dose 1.0) |
| FEMALE | 88.0 ± 4.82 | 98.5 ± 5.62 | 109.3 ± 4.5 | 108.6 ± 3.74^{a} | 77.2 ± 3.14^{b} | 69.2 ± 2.5^{b} |
| MALE | 69.3 ± 2.5 | 86.2 ± 3.76 | 90.7 ± 4.73 | 90.0 ± 1.7^{a} | 65.0 ± 2.28^{b} | 69.6 ± 2.73^{b} |

( X̅ ± S.E.M)
(^{a,b,c}p < 0.01)

TABLE 11
Triglycerides level (mg/dl) in the blood serum of rats

| | | | | | 120 day + | 120 day + |
|---|---|---|---|---|---|---|
| | | | | 120 day + | AKG | AKG |
| | 0 day | 30 day | 60 day | H₂O | (dose 0.1) | (dose 1.0) |
| FEMALE | 73.8 ± 8.1 | 146.8 ± 16.11 | 196.7 ± 30.43 | 183.9 ± 3.97^{a} | 111.7 ± 1.85^{b} | 142.0 ± 2.39^{c} |
| MALE | 70.0 ± 11.47 | 153.7 ± 13.79 | 180.0 ± 17.72 | 182.4 ± 4.54^{a} | 102.2 ± 10.89^{b} | 119.6 ± 17.35^{b} |

( X̅ ± S.E.M)
(^{a,b,c}p < 0.01)

TABLE 12
LDL level (mg/dl) in the blood serum of rats

| | | | | | 120 day + | 120 day + |
|---|---|---|---|---|---|---|
| | | | | 120 day + | AKG | AKG |
| | 0 day | 30 day | 60 day | H₂O | (dose 0.1) | (dose 1.0) |
| FEMALE | 13.0 ± 2.89 | 19.7 ± 5.44 | 32.0 ± 2.86 | 30.8 ± 1.31^{a} | 0.20 ± 0.015^{b} | 0.20 ± 0.019^{b} |
| MALE | 14.0 ± 1.34 | 17.7 ± 3.58 | 30.3 ± 1.84 | 32.5 ± 5.42^{a} | 5.4 ± 2.48^{b} | 10.2 ± 2.31^{b} |

( X̅ ± S.E.M)
(^{a, b, c}p < 0.001)

TABLE 13
HDL level (mg/dl) in the blood serum of rats

| | | | | | 120 day + | 120 day + |
|---|---|---|---|---|---|---|
| | | | | 120 day + | AKG | AKG |
| | 0 day | 30 day | 60 day | H₂O | (dose 0.1) | (dose 1.0) |
| FEMALE | 60.3 ± 3.9 | 49.2 ± 3.72 | 46.3 ± 4.64 | 37.7 ± 1.65 | 39.4 ± 0.68 | 40.2 ± 1.59 |
| MALE | 41.7 ± 3.36 | 37.5 ± 3.49 | 28.7 ± 1.55 | 24.5 ± 1.99^{a} | 39.0 ± 1.67^{b} | 35.6 ± 2.32^{b} |

( X̅ ± S.E.M)
(^{a,b,c}p < 0.05)

TABLE 14
Body weight (g) of rats( X̅ ± S.E.M)(^{a,b,c}p < 0.05)

| | | | | | 120 day + | 120 day + |
|---|---|---|---|---|---|---|
| | | | | 120 day + | AKG | AKG |
| | 0 day | 30 day | 60 day | H₂O | (dose 0.1) | (dose 1.0) |
| FEMALE | 269.7 ± 6.48 | 304.6 ± 4.84 | 330.0 ± 7.15 | 356.7 ± 5.77 | 355.6 ± 6.62 | 350.8 ± 9.7 |
| MALE | 371.3 ± 3.27 | 445.9 ± 10.7 | 447.3 ± 3.01 | 512.2 ± 7.96^{a} | 519.2 ± 7.39^{a} | 462.1 ± 0.9^{b} |

### EXAMPLE 3

### Experiment III

Studies were conducted on volunteers with a relatively high level of cholesterol, 194 mg/dl and 190 mg/dl respectively. Chewable tablets were prepared each containing 1.28 g of calcium alpha-ketoglutarate (corresponding to 1 g of alpha-ketoglutaric acid (AKG) and 0.28 g of calcium) and administered orally to the volunteers. The experiment lasted for the total of 42 days. From day 1 to day 28, patients took two tablets of AKG three times a day. During the experiment no quantitative or qualitative restrictions regarding the diet were introduced. In the period from day 1 to day 28 the patients' lipid profile was checked every seven days in order to determine the concentration of cholesterol, fractions of LDL and HDL and triglycerides. The administration of AKG tablets was discontinued for 14 days after a 28 day period. Subsequent measurements of the lipid profile were taken on 42^{nd} day of the experiment.

The results are reported in Tables 15 to 18 below.

TABLE 15
Total cholesterol level (mg/dl)

| Week of | | | | | | |
|---|---|---|---|---|---|---|
| follow-up | 1 ^{st} | 2 ^{nd} | 3 ^{th} | 4 ^{th} | | 6 ^{th} |
| examination | week | week | week | week | | week |
| Volunteer 1 | 194 | 196 | 171 | 157 | 2 weeks | 197 |
| (aged 38- | | | | | without | |
| male) ) | | | | | AKG | |
| Volunteer 2 | 190 | 213 | 199 | 189 | | 215 |
| (aged 38- | | | | | | |
| male) | | | | | | |

TABLE 16
HDL level (mg/dl)

| Week of | | | | | | |
|---|---|---|---|---|---|---|
| follow-up | 1^{st} | 2^{nd} | 3^{th} | | | 6^{th} |
| examination | week | week | week | 4^{th} week | | week |
| Volunteer 1 | 45 | 41 | 42 | 48 | 2 weeks | 33 |
| (aged 38- | | | | | without | |
| male) ) | | | | | AKG | |
| Volunteer 2 | 35 | 44 | 40 | 42 | | 40 |
| (aged 38- | | | | | | |
| male) | | | | | | |

TABLE 17
LDL level (mg/dl)

| Week of | | | | | | |
|---|---|---|---|---|---|---|
| follow-up | 1^{st} | 2^{nd} | 3^{th} | | | 6^{th} |
| examination | week | week | week | 4^{th} week | | week |
| Volunteer 1 | 110 | 94 | 75 | 57 | 2 weeks | 88 |
| (aged 38- | | | | | without | |
| male) ) | | | | | AKG | |
| Volunteer 2 | 131 | 148 | 119 | 110 | | 131 |
| (aged 38- | | | | | | |
| male) | | | | | | |

TABLE 18
Triglycerides level (mg/dl)

| Week of | | | | | | |
|---|---|---|---|---|---|---|
| follow-up | 1^{st} | 2^{nd} | 3^{th} | | | 6^{th} |
| examination | week | week | week | 4^{th} week | | week |
| Volunteer 1 | 197 | 304 | 269 | 259 | 2 weeks | 375 |
| aged 38- | | | | | without | |
| male | | | | | AKG | |
| Volunteer 2 | 122 | 101 | 199 | 187 | | 220 |
| (aged 38- | | | | | | |
| male) | | | | | | |

Discussion

The application of ovariectomy (OVX) commonly regarded as a model approach simulating the symptoms observed in the course of postmenopausal syndrome, led to an increase in the concentration of cholesterol in the blood plasma of the rats. The application of AKG in the dose of 0.01 during 60 days in OVX females (Experiment I-1) resulted in statically significant decrease (p=0.04) in the concentration of the entire cholesterol in these animals reaching the level observed in animals from the control group. A similar tendency was observed in rats that received AKG in dose 0.1 (Experiment I-1). The effects of AKG were more visible in females with a prolonged lack of ovarian hormones (Experiment I-2). Both doses, 0.01 and 0.1, of alpha-ketoglutarate caused a statistically significant decrease in the concentration of the entire cholesterol in the animals under analysis in OVX and SHO groups. Also the application of dose 1 of alpha-ketoglutarate decreased the concentration of the entire cholesterol in the females after ovariectomy relative to the control group both in the experiments involving a short-term and long-term absence of the activity of ovarian hormones (Experiments I-1 and I-2).

During Experiment II fodder enriched with 1% of cholesterol and 10% of the lard to induct hypercholesterolaemia was used. The analysis of the cholesterol profile after 30 days of the experiment indicated statistically increase in the concentration of triglycerides relative to the control group as at day 0 both in males and in females. Moreover, a significant increase in the entire cholesterol concentration was determined in males. It should be emphasized that in females and in males alike a falling tendency with respect to concentration of HDL and a matching increase in the concentration of LDL was observed after a 60 day period of feeding with the experimental fodder.

From day 61 for 60 consecutive days AKG solutions in doses 0.1 and 1 were administered. The analysis of a lipid profile performed on day 120 of the experiment indicated a statistically significant decrease in the entire cholesterol, LDL fraction and triglycerides (p<0.05) in the blood plasma of females and males. A simultaneous increase in the concentration of HDL in the tested animals was observed and statistically confirmed (p<0.05).

Experiment III consisted in examining two volunteers with a relatively high level of the entire cholesterol, LDL fraction and triglycerides. After 28 days of receiving specially prepared tablets containing 1 g of AKG (2 tablets 3 time a day) a decrease in the concentration of the entire cholesterol and LDL fraction was observed. No significant effect on the concentration of HDL was observed. The concentration of HDL was somewhat lowered during the second and third week of the experiment, and on day 28 its level 10 matched the original level. From day 1 to day 28 of the experiment an increase in the concentration of triglycerides was observed. The concentration of the entire cholesterol and LDL fraction which decreased with the application of alpha-ketoglutarane increased after a 14 day break in administering the preparation. A significant increase in the concentration of triglycerides was also observed. At the same time a statistically significant decrease in the concentration of HDL fraction of cholesterol was determined.

1-8. (canceled)

9. The use of at least one member selected from the group consisting of alpha-ketoglutaric acid and pharmaceutically acceptable salts thereof and pharmaceutically accepted physical mixtures of alpha-ketoglutaric acid or a pharmaceutically acceptable salt thereof and at least one amino acid, for the manufacture of a pharmaceutical preparation or a food or feed supplement for the treatment or prophylaxis of a condition of increased plasma levels of at least one member selected from a group consisting of low density lipids (LDL) and triglycerides or for the promotion of high density lipid (HDL) production in vertebrates, such as birds and mammals, including man.

10. The use of alpha-ketoglutaric acid or pharmaceutically acceptable salt thereof for the manufacture of a pharmaceutical preparation or a food or a feed supplement for the treatment of prophylaxis of a condition of increased plasma levels of low density lipids (LDL) in vertebrates, such as birds and mammals, including man.

11. The use of alpha-ketoglutaric acid or pharmaceutically acceptable salt thereof for the manufacture of a pharmaceutical preparation or a food or a feed supplement for the treatment of prophylaxis of a condition of increased plasma levels of triglycerides in vertebrates, such as birds and mammals, including man.

12. The use of alpha-ketoglutaric acid or pharmaceutically acceptable salt thereof for the manufacture of a pharmaceutical preparation or a food or a feed supplement for the promotion of high density lipid (HDL) production in vertebrates, such as birds and mammals, including man.

13. Use according to claim 9, wherein alpha-ketoglutaric acid or an alkali or alkaline earth metal salt thereof or a combination thereof is used.

14. Use according to claim 10, wherein alpha-ketoglutaric acid or an alkali or alkaline earth metal salt thereof or a combination thereof is used.

15. Use according to claim 11, wherein alpha-ketoglutaric acid or an alkali or alkaline earth metal salt thereof or a combination thereof is used.

16. Use according to claim 12, wherein alpha-ketoglutaric acid or an alkali or alkaline earth metal salt thereof or a combination thereof is used.

17. Use according to claim 13, wherein sodium alpha-ketoglutarate is used.

18. Use according to claim 14, wherein sodium alpha-ketoglutarate is used.

19. Use according to claim 15, wherein sodium alpha-ketoglutarate is used.

20. Use according to claim 16, wherein sodium alpha-ketoglutarate is used.

21. Method for the treatment or prophylaxis of a condition of increased plasma levels of at least one member selected from the group consisting of low density lipids (LDL) and triglycerides in birds and mammals, including man, which method comprises administering to a subject in needs for such treatment or prophylaxis of an effective plasma level lowering amount of at least one member selected from the group consisting of alpha-ketoglutaric acid and pharmaceutically acceptable salt thereof and pharmaceutically accepted physical mixtures of alpha-ketoglutaric acid or a pharmaceutically acceptable salt thereof and at least one amino acid.

22. Method for the promotion of high density lipid (HDL) production in a bird or a mammal, including man, which method comprises administering to said bird or mammal an effective plasma HDL level increasing amount of at least one member selected from the group consisting of alpha-ketoglutaric acid, and pharmaceutically acceptable salts thereof and pharmaceutically accepted physical mixtures of alpha-ketoglutaric acid an other amino acids or a pharmaceutically acceptable salt thereof and at least one amino acid.

23. Method according to claim 21, wherein alpha-ketoglutaric acid or an alkali or alkaline earth metal salt thereof or a combination thereof is administered.

24. Method according to claim 22, wherein alpha-ketoglutaric acid or an alkali or alkaline earth metal salt thereof or a combination thereof is administered.

25. Method according to claim 23, wherein sodium alpha-ketoglutarate is administered.

26. Method according to claim 24, wherein sodium alpha-ketoglutarate is administered.

27. Method according to claim 21, wherein a pharmaceutical preparation or a food or feed supplement comprising the active principle or principles in administered.

28. Method according to claim 22, wherein a pharmaceutical preparation or a food or feed supplement comprising the active principle or principles in administered.

29. Method according to claim 23, wherein a pharmaceutical preparation or a food or feed supplement comprising the active principle or principles in administered.

30. Method according to claim 24, wherein a pharmaceutical preparation or a food or feed supplement comprising the active principle or principles in administered.

31. Method according to claim 25, wherein a pharmaceutical preparation or a food or feed supplement comprising the active principle or principles in administered.

32. Method according to claim 26, wherein a pharmaceutical preparation or a food or feed supplement comprising the active principle or principles in administered.
